(19) United States
(12) Patent Application Publication
(10) Pub. No.: US 2006/0243640 A1
(43) Pub. Date: Nov. 2, 2006

(54) Process for the production of compression ignition engine, gas turbine, and fuel cell fuel and compression ignition engine, gas turbine, and fuel cell fuel by said process

(76) Inventors: Luis Pablo Dancuart, Vaalpark (ZA); Delanie Lamprecht, Vanderbijlpark (ZA); Ian Stradling Myburgh, Vanderbijlpark (ZA)

Correspondence Address: HAHN AND MOODLEY, LLP, P.O. BOX 52050, MINNEAPOLIS, MN, 55402, US

(21) Appl. No.: 11/406,016

(22) Filed: Apr. 18, 2006

Related U.S. Application Data
(63) Continuation of application No. PCT/ZA04/00125, filed on Oct. 14, 2004.

(30) Foreign Application Priority Data
Oct. 17, 2003 (ZA) ZA20038080

(51) Int. Cl. C10L 1/04; C07C 27/06

(52) U.S. Cl. 208/15; 518/702

(57) ABSTRACT

The invention provides a Fischer-Tropsch derived compression ignition engine, gas turbine, and fuel cell fuel which is interchangeably useable in compression ignition engines, gas turbines, and fuel cells, said fuel selected from a substantially C5 to C9 cut, a substantially C5 to C9 cut blended with a substantially C9 to C14 cut, a substantially C5 to C9 cut blended with a substantially C9 to C14 cut and a substantially C14 to C22 cut, and a substantially C5 to C9 cut blended with a substantially C14 to C22 cut. The invention extends to a process for preparing said fuel and the use of such a fuel in a CI engine, and HCCI engine, a turbine, and/or a fuel cell.

### CROSS RELATED APPLICATION(S)

This application is a continuation of PCT Patent Application PCT/ZA2004/000125 filed Oct. 14, 2004 and published on Apr. 4, 2005 as WO 2005/035695, which claims priority to ZA 2003/8080 filed Oct. 17, 2003 and US Provisional Application 60/512330 filed Oct. 17, 2003.

### FIELD OF THE INVENTION

The invention relates to the production of compression ignition engine, gas turbine, and fuel cell fuels.

### BACKGROUND TO THE INVENTION

In this specification, the term “multipurpose hydrocarbonaceous energy sources” is abbreviated to MES and is used in both the singular and the plural.

The term MES thus encompasses compression ignition engine, gas turbine, and fuel cell fuels.

An MES usable in gas turbines, compression ignition (CI) engines, including Homogeneous Charge Compression Ignition (HCCI) systems or fuel cells is an attractive option for many energy users, especially for those operating in remote stranded locations where a single form of supply of energy is required and simplified logistics are necessary. These entities include users in many classes of human activity.

U.S. Pat. No. 6,475,375, discloses the process for the production of a synthetic naphtha fuel usable in CI engines. This patent, however, does not contemplate the use of such a fuel as an MES having broader application other than use thereof in a CI engine. Thus, the disclosure in this patent does not provide any indication of how the problems associated with the production of an MES may be overcome or what characteristics or properties such an MES should have.

A synthetic multi-purpose fuel useful as a fuel cell fuel, diesel engine fuel, gas turbine engine fuel and furnace or boiler fuel are disclosed in PCT WO 01/59034. The multi-purpose fuel produced ranged from C9 to C22.

The inventor has now identified a need and a process for at least partially satisfying such an MES need.

The Fischer-Tropsch (FT) process is a well known process in which carbon monoxide and hydrogen are reacted over an iron, cobalt, nickel or ruthenium containing catalyst to produce a mixture of straight and branched chain hydrocarbons ranging from methane to waxes with molecular masses above 1400 and smaller amounts of oxygenates. The feed for the FT process may be derived from coal, natural gas, biomass or heavy oil streams. The term Gas-to-Liquid (GTL) process refers to schemes based on natural gas, which is mainly methane, to obtain the synthesis gas, and its subsequent conversion using in most instances an FT process. The quality of the GTL FT synthetic products is essentially the same obtainable from the FT process here defined once the synthesis conditions and the product work-up are defined.

The complete process can include gas reforming which converts natural gas to synthesis gas (H₂ and CO) using well-established reforming technology. Alternatively, synthesis gas can also be produced by gasification of coal or suitable hydrocarbonaceous feedstocks like petroleum based heavy fuel oils. The synthesis gas is then converted into synthetic hydrocarbons. The- process can be effected using, among others, a fixed-bed tubular reactor or a three-phase slurry reactor. FT products include waxy hydrocarbons, light liquid hydrocarbons, a small amount of unconverted synthesis gas and a water-rich stream. The waxy hydrocarbon stream and, almost always, the light liquid hydrocarbons are then upgraded in the third step to synthetic fuels such as diesel, kerosene and naphtha. Heavy species are hydrocracked and olefins and oxygenates are hydrogenated to form a final product that is highly paraffinic. Hydrocracking and hydrogenation processes belong to the group sometimes generally named hydroconversion processes.

### SUMMARY OF THE INVENTION

According to a first aspect of the invention, there is provided a multipurpose carbonaceous energy source (MES fuel) which is a compression ignition engine, gas turbine, and fuel cell fuel which fuel is interchangeably useable in compression ignition engines, gas turbines, and fuel cells, said energy source selected from:
a substantially C5 to C9 cut blended with a substantially C9 to C14 cut, said blend having an H:C molar ratio from 2.18 to 2.24;
a substantially C5 to C9 cut blended with a substantially C9 to C14 cut and a substantially C14 to C22 cut, said blend having an H:C ratio from 2.12 to 2.18; and
a substantially C5 to C9 cut blended with a substantially C14 to C22 cut, said blend having an H:C molar ratio from 2.13 to 2.19.

The MES fuel options as defined in this invention are summarised in Table 1.

TABLE 1
MES Fuels

| | | Carbon Number Range | | | H:C | |
|---|---|---|---|---|---|---|
| | | Cut A | Cut B | Cut C | Ratio | CO₂ Emissions |
| MES | Fuel Cut | C5-C9 | C9-C14 | C14-C22 | Molar | g CO₂/g fuel |
| 1 | C5-C9 | X | | | 2.29 | 3.080 |
| 2 | C5-C14 | X | X | | 2.20 | 3.098 |
| 3 | C5-C22 | X | X | X | 2.14 | 3.111 |
| 4 | C5-C9 & | X | | X | 2.17 | 3.105 |
| | C14-C22 | | | | | |

The MES fuel may, when combusted, have a CO₂ emission below 3.115 g CO₂/g fuel combusted.

One or more of the C5 to C9, C9 to C14, and C14 to C22 cuts may be synthetic in origin.

One or more of the C5 to C9, C9 to C14, and C14 to C22 cuts may be Fischer-Tropsch process in origin.

The MES Fuel may be a partially or totally synthetic fuel.

The MES Fuel may be a Fischer-Tropsch process derived fuel.

According to a second aspect of the invention, there is provided a process for the production of synthetic multipurpose carbonaceous energy source (MES fuels) which is a compression ignition engine, gas turbine, and fuel cell fuel, which fuel is interchangeably useable in compression ignition engines, gas turbines, and fuel cells, said process including the steps of:
a) oxidising a carbonaceous material to form a synthesis gas;
b) reacting said synthesis gas under Fischer-Tropsch reaction conditions to form Fischer-Tropsch reaction products;
c) fractionating the Fischer-Tropsch reaction products to form one or more MES blending components selected from the group including:
   A. a C5 to C9 cut;
   B. a C9 to C14 cut; and
   C. a C14 to C22 cut; and
d) using said blending components in the production of the MES, provided that where at least one of the blending components is a blending component in the C9 to C14 or in the C14 to C22 boiling range then at least two blending components are used in the production of the MES, one of which is the C5 to C9 cut.

The C5 to C9 cut may be a light hydrocarbon blend, typically in the 35-160° C. distillation range.

The C9 to C14 cut may be a medium hydrocarbon blend, typically in the 155-250° C. distillation range.

The C14 to C22 cut may be a heavy hydrocarbon blend, typically in the 245-360° C. distillation range.

To obtain the MES fuels of Table 1, the blending components A, B and C, as described above, may be blended in a volumetric ratio of A:B:C of:

1.0:0.0:0.0 for MES 1

and

1.2:1.0:0.0 for MES 2

1.8:1.0:2.3 for MES 3

1.0:0.0:2.1 for MES 4

to

1.0:1.2:0.0 for MES 2

1.0:1.2:1.8 for MES 3

1.0:0.0:1.5 for MES 4

To obtain the MES fuels of Table 1, the blending components A, B and C may be blended in a volumetric ratio of A:B:C, wherein:

A may be from 1 to 2;

B may be from 0 to 1.5; and

C may be from 0 to 2.5.

One or more of the blending components may be hydroconverted.

Thus, the MES may be a blend of both hydroconverted and unhydroconverted blending components.

The MES may be a product of one or more of only unhydroconverted blending components.

The MES may be a product of one or more only hydroconverted blending components.

The Fischer-Tropsch process of step b) may be the Sasol Slurry Phase Distillate™ process.

The carbonaceous material of step a) may be a natural gas stream, a natural gas derivatives stream, a petroleum gas stream, a petroleum gas derivatives stream, a coal stream, a waste hydrocarbons stream, a biomass stream, and in general any carbonaceous material stream.

Optionally, hydrogen may be separated from the synthesis gas either during or after step a).

This hydrogen may be used in the hydroconversion of FT primary products, namely FT condensate and FT wax.

Table 2 below gives a typical composition of the FT condensate and FT wax fractions.

TABLE 2
Typical Fischer-Tropsch product after separation
into two fractions (vol % distilled)

| | | FT Condensate | FT Wax |
|---|---|---|---|
| | | (<270° C. fraction) | (>270° C. fraction) |
| C₅-160° | C. | 44 | 3 |
| 160-270° | C. | 43 | 4 |
| 270-370° | C. | 13 | 25 |
| 370-500° | C. | | 40 |
| >500° | C. | | 28 |

In one embodiment of the invention, the hydroconverted products are fractionated in a common distillation unit where at least three blending components are recovered:
(1) a light hydrocarbon blend, typically in the 35-160° C. ASTM D86 distillation range, i.e. C5 to C9;
(2) a medium hydrocarbon blend, typically in the 155-250° C. ASTM D86 distillation range, i.e. C9to C14; and
(3) a heavy hydrocarbon blend, typically in the 245-360° C. ASTM D86 distillation range, i.e. C14 to C22.

However, in other embodiments, the FT condensate and FT wax are blended together before being fractionated into the blending components.

In some embodiments the FT condensate is transferred directly to the products fractionator without any hydroconversion stage.

When processing using this approach, the MES products benefit from the synergy of the composition and quality of the wax and condensate fractions.

MES fuels of the invention meet the fuel requirements of many classes of energy conversion systems including gas turbines, CI engines, including HCCI systems and fuel cells.

The MES compositions may have the following properties which make it suitable for fuel cells, gas turbine engine and CI engines (as shown in Table 3 below):

TABLE 3
Quality of the Multipurpose Energy Sources

| | | Light HC | Medium | Heavy HC | | | |
|---|---|---|---|---|---|---|---|
| | | Blend | HC Blend | Blend | MES-1 | MES-2 | MES-3 |
| Yield (est.) | wt % | 28% | 25% | 47% | 28% | 53% | 100% |
| Density @ 15° C. | kg/l | 0.690 | 0.752 | 0.782 | 0.690 | 0.723 | 0.747 |
| Cetane Number (IQT) | | 44 | 64 | >72 | 44 | 60 | 64 |
| Sulphur | wt ppm | <1 | <1 | <1 | <1 | <1 | <1 |
| ASTM D86 Distillation range | ° C. | 35-160 | 155-250 | 245-360 | 35-160 | 35-250 | 35-360 |
| Cold Filter Plugging Point | ° C. | <−30 | <−30 | −12 | <−30 | <−30 | <−30 |
| Freezing point | ° C. | <−60 | −48 | −9 | <−60 | <−60 | <−60 |
| Flash Point | ° C. | <0 | 50 | 114 | <0 | <0 | 14 |
| Aromatics | wt % | 1.0-2.0 | 0.5-1.0 | <0.5 | 1.0-2.0 | 1.0-1.5 | 0.5-1.0 |
| Biodegradabily Test | | pass | pass | pass | pass | pass | pass |
| Thermal stability (Octel | Visual | 1 | 1 | 1 | 1 | 1 | 1 |
| F21-61) | rating (relative | (Excellent) | (Excellent) | (Excellent) | (Excellent) | (Excellent) | (Excellent) |
| | stability) | | | | | | |
| Oxidation Stability | mg/100 ml | 0.1 | 0.1 | 0.2 | 0.1 | 0.1 | 0.1 |
| Viscosity @ 40° C. | cSt | 0.98 | 1.14 | 3.3 | 0.98 | 1.10 | 1.34 |

HC = Hydrocarbon

High Cetane Number: Fuels with a high cetane number ignite quicker and hence exhibit a milder uncontrolled combustion because the quantity of fuel involved is less. A reduction of the uncontrolled combustion implies an extension of the controlled combustion, which results in better air/fuel mixing and more complete combustion with lower NOx emissions and better cold start ability. The shorter ignition delay implies lower rates of pressure rise and lower peak temperatures and less mechanical stress.

The cetane number of the MES compositions was determined according to ASTM D613 test method and an Ignition Quality Tester (IQT-ASTM D6890).

Near Zero-Sulphur Content: The sulphur content was determined according to the ASTM D5453 test method. The less than 1 ppm sulphur present in the MES compositions not only make the components suitable for a fuel cell reformer catalyst, but also contribute to the lower exhaust emission in engines, such as CI engines. The less than 1 ppm sulphur present in the MES composition either ensure compatible with certain exhaust catalyst devises or give improved compatibility with other.

Good Cold Flow Properties: Cold Filter Plugging Point (CFPP) is the lowest temperature at which the fuel can pass through a standard test filter under standard conditions (requires more than 1 minute for 20 ml to pass through a 45-μm filter). This test is done accordingly to the Institute of Petroleum IP 309 method or equivalent. Inadequate cold flow performance will lead to difficulties with starting and blockage of CI engine fuel filters under cold weather conditions.

Freezing point is one of the physical properties used to quantitatively characterise gas turbine engine fuel fluidity. The low freezing point, determined in accordance with the automated ASTM 5901 test method, or equivalent, can be attributed to the more than 60 mass % iso-paraffins present in MES compositions.

Excellent Thermal and Oxidation Stability: The thermal stability of the MES compositions was determined according to the Octel F21-61 test method where a visual rating was used to describe the relative stability. The FT products lead to significantly less carbon deposition on the fuel cell reformer catalyst than would be expected from a conventional diesel type feedstock under comparative reaction conditions.

Oxygen stability is tested through the calculation of the amount of insolubles formed in the presence of oxygen. It measures the fuel's resistance to degradation by oxygen by the ASTM D2274 test method or equivalent. The MES compositions are stable in the presence of oxygen with the formation of insolubles of less than 0.2 mg/100 ml.

High Hydrogen To Carbon Content: The highly paraffinic nature of the FT products and very low aromatic concentration contribute to the high H:C ratios of the MES compositions.

In Table 1, four illustrative MES formulations are shown which have been found compatible with their proposed use in gas turbines, CI engines, including HCCI systems and fuel cells. The expected quality and estimated yields of the MES formulations of Table 1 are presented in Table 3.

The MES compositions may be suitable for use in fuel cells, gas turbine engine and CI engines, including HCCI systems as they contain FT reaction derived products which are highly saturated with less than 2 volume % olefins, have ultra-low levels of sulphur with an almost zero aromatic hydrocarbon content, high linearity, high hydrogen to carbon ratio, very good cold flow properties, and high cetane number.

Lower reformer temperatures in fuel cells are required with the use of FT naphtha, kerosene or diesel. The FT products lead to significantly less carbon deposition on the catalyst than would be expected from a conventional diesel type feedstock under comparative reaction conditions and produce more steam. The MES components have good cold flow properties as well as a high cetane number because of the predominantly mono-, and to a lesser extent other, branched forms of the paraffins which make these components suitable for application in gas turbine engines, CI engines, including HCCI systems and fuel cells.

The highly paraffinic related properties such as high H:C ratio, high cetane number and low density together with virtually zero-sulphur and very low aromatics content give the FT products their very good emission performance

### DESCRIPTION OF THE DRAWINGS

The invention will now be described by way of non-limiting example only with reference to the accompanying drawings. In the drawings,

FIG. 1 shows a flow sheet for a process for the production of a fuel of the invention;

FIG. 2 shows a flow sheet for an alternative process to that in FIG. 1 but based on Natural gas;

FIG. 3 shows a flow sheet for a process using high molecular mass feedstocks; and

FIG. 4 shows a flow sheet for a process such as that of FIG. 3 using use of coal, biomass or heavy oil as feedstock.

### PROCESS DESCRIPTION

This invention includes four possible processes for the production of MES components i.e. components for Fischer-Tropsch derived compression ignition engine, gas turbine, and fuel cell fuel which is interchangeably useable in compression ignition engines, gas turbines, and fuel cells. Two of them are based in the use of natural gas as feed and, the other two make use of any hydrocarbonaceous feedstock possible of been gasified. Therefore, feeds for the latter include coal, waste, biomass and heavy oil streams.

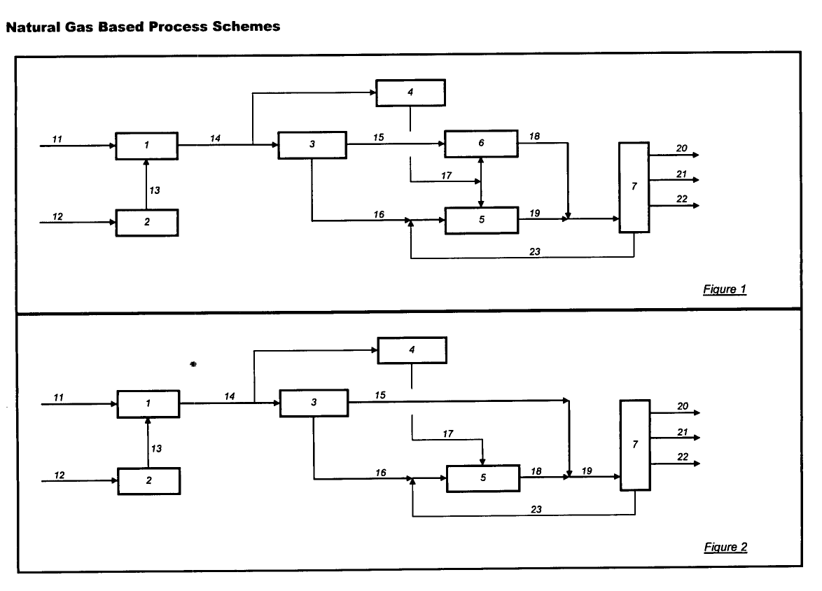

The first process matter of this invention, presented in FIG. 1, makes use of natural gas 11 which is converted to synthesis gas at suitable process conditions in reformer 1. The reforming reaction makes use of oxygen 13 obtained from an air separation step 2 from atmospheric air 12. Water in the form of steam can also be used in the reforming process.

Syngas 14 from the reformer stage is converted in FT unit 3 to synthetic hydrocarbons including at least two liquid streams, as well as a gas stream and reaction water not shown. A portion of the syngas might be derived from the hydrogen separation plant 4 where a hydrogen rich stream 17 is produced for use in hydroconversion. Alternatively, hydrogen can be produced in an independent facility and transferred as stream 17.

The light synthetic hydrocarbons stream 15, sometimes named FT Condensate, includes paraffins, olefins and some oxygenates, mostly alcohols. This stream is transferred to hydrotreating unit 6 where olefins and oxygenates are hydrogenated into, mostly, the corresponding paraffin hydrocarbons. The process is completed at conditions such that the average carbon number of the feed remains essentially unchanged in hydrotreated product 18.

The heavy synthetic hydrocarbons 16, sometimes named FT Wax, has a similar chemical composition as that of the lighter stream 15; however, under normal processing these species are solid at room temperature. This stream is transferred to the hydroconversion unit 5, preferably a hydrocracker system, where (1) olefins and oxygenates are hydrogenated to the corresponding paraffins which in turn and together with the original long chain paraffins (2) undergo cracking reactions resulting in a significant reduction of its average carbon number compared with that of the feed. The resulting hydrocracked product 19 is a mixture of normal and iso-paraffins.

The combined hydroconverted products 18 and 19 are fractionated in distillation unit 7 resulting in at least four process streams. Stream 20 is a light hydrocarbon blend, typically in the 35-160° C. ASTM D86 distillation range. Stream 21 is a medium hydrocarbon blend, typically in the 155-250° C. ASTM D86 distillation range. Stream 22 is a heavy hydrocarbon blend, typically in the 245-360° C. ASTM D86 distillation range. Stream 23 includes unconverted species whose boiling points are above 360° C. and is recycled to the hydrocracker to increase the production of the valuable species. The separation process also results in collecting a gas stream—not shown.

The MES products are produced using these streams on their own or in blends as shown in Table 1 above.

An alternative second process scheme based on natural gas is presented in FIG. 2. From a process standpoint it differs from the one described before in that the light synthetic hydrocarbons 15 is not hydrotreated. Instead it is blended with the hydrocracked product 18. The resulting stream 19 is fractionated then in distillation unit 7 resulting in products 20-22 similar to those above described. However, while these products can be used in the same blends, they include some olefins and oxygenates in their composition.

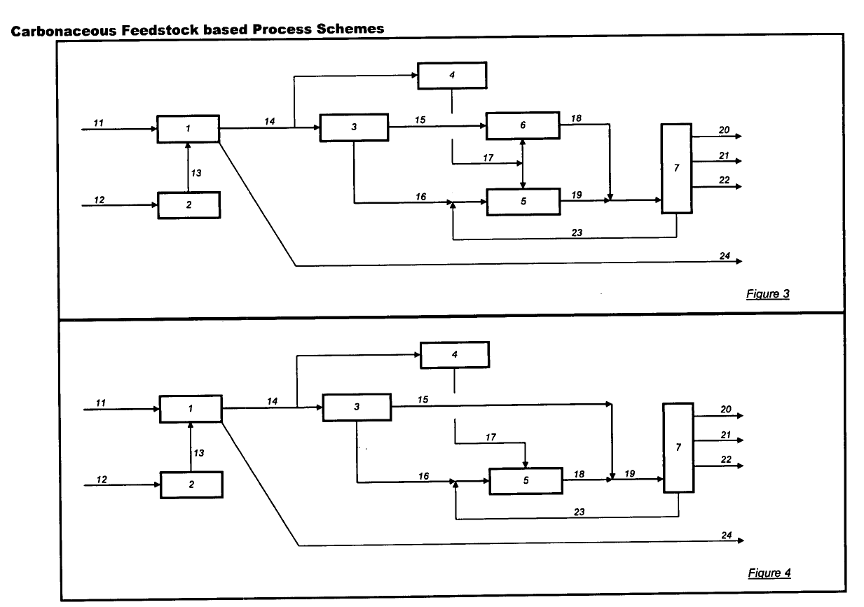

Using alternative high molecular mass feedstocks this invention provides the process scheme shown in FIG. 3. This concept makes use of coal, biomass or heavy oil which in the form of stream 11 is converted to synthesis gas at suitable process conditions in gasifier 1. The gasification process makes use of oxygen 13 obtained from an air separation step 2 from atmospheric air 12. Water in the form of steam can also be used in the process. This process is then substantially similar to the one discussed before with reference to FIG. 1. However, and as an additional stream, some liquids are produced during the gasification process and separated as stream 24. These might be recovered as a product or recycled to the gasifier to enhance production of the valuable streams. Other than this, process units and streams in FIG. 3 correspond to those in FIG. 1 and its associated process description

Finally, and as an alternative to this concept, it is provided a fourth process scheme similar in essence to the second option discussed here above. As the one just discussed, this makes use of coal, biomass or heavy oil as feedstock and makes use of gasifier 1 as described in the previous paragraph. This process is then substantially similar to the one discussed before with reference to FIG. 2. However, and as an additional stream, some liquids are produced during the gasification process and separated as stream 24. These might be recovered as a product or recycled to the gasifier to enhance production of the valuable streams. Other than this, process units and streams in FIG. 4 correspond to those in FIG. 2 and its associated process description.

1. A process for the production of Fischer-Tropsch derived compression ignition engine, gas turbine, and fuel cell fuel which is interchangeably useable in compression ignition engines, gas turbines, and fuel cells, said process including the steps of:
a) oxidising a carbonaceous material to form a synthesis gas;
b) reacting said synthesis gas under Fischer-Tropsch reaction conditions to form Fischer-Tropsch reaction products;
c) hydroconverting said products;
d) fractionating the hydroconverted Fischer-Tropsch reaction products to form at least a C5 to C9 cut as a blending component A and one or more blending components selected from the group including:
B. a C9 to C14 cut; and
C. a C14 to C22 cut; and
e) using said blending components in the production of said fuel.

2. A process as claimed in claim 1, wherein the C5 to C9 cut is a light hydrocarbon blend having a 35-160° C. distillation range.

3. A process as claimed in claim 1, wherein the C9 to C14 is a medium hydrocarbon blend having a 155-250° C. distillation range.

4. A process as claimed in claim 1, wherein the C14 to C22 cut is a heavy hydrocarbon blend having a 245-360° C. distillation range.

5. A process as claimed in claim 1 , wherein fuels are produced by the use of blending components A, B, and C blended in a volumetric ratio of A:B:C wherein:
A may be from 1 to 2;
B may be from 0 to 1.5; and
C may be from 0 to 2.5.

6. A process as claimed in claim 1, wherein the Fischer-Tropsch process of step b) is a slurry phase distillate process.

7. A process as claimed in claim 5, wherein the Fischer-Tropsch process of step b) is a slurry phase distillate process.

8. A process as claimed in claim 1, wherein the carbonaceous material of step a) is a natural gas stream, a natural gas derivatives stream, a petroleum gas stream, a petroleum gas derivatives stream, a coal stream, a waste hydrocarbons stream, a biomass stream, and in general any carbonaceous material stream.

9. A process as claimed in claim 5, wherein the carbonaceous material of step a) is a natural gas stream, a natural gas derivatives stream, a petroleum gas stream, a petroleum gas derivatives stream, a coal stream, a waste hydrocarbons stream, a biomass stream, and in general any carbonaceous material stream.

10. A process as claimed in claim 6, wherein the carbonaceous material of step a) is a natural gas stream, a natural gas derivatives stream, a petroleum gas stream, a petroleum gas derivatives stream, a coal stream, a waste hydrocarbons stream, a biomass stream, and in general any carbonaceous material stream.

11. A process as claimed in claim 7, wherein the carbonaceous material of step a) is a natural gas stream, a natural gas derivatives stream, a petroleum gas stream, a petroleum gas derivatives stream, a coal stream, a waste hydrocarbons stream, a biomass stream, and in general any carbonaceous material stream.

12. A process as claimed in claim 1, wherein the hydroconverted products from step c) are fractionated in step d) in a common distillation unit where at least three blending components are recovered:
(1) a light hydrocarbon blend;
(2) a medium hydrocarbon blend; and
(3) a heavy hydrocarbon blend.

13. A compression ignition engine, gas turbine, and fuel cell fuel which is interchangeably useable in compression ignition engines, gas turbines, and fuel cells, said fuel comprising a substantially C5 to C9 hydroconverted cut blended with a substantially C9 to C14 hydroconverted cut, which cuts have the Fischer-Tropsch process as their origin, said blend having an H:C molar ratio from 2.18 to 2.24.

14. A compression ignition engine, gas turbine, and fuel cell fuel which is interchangeably useable in compression ignition engines, gas turbines, and fuel cells, said fuel comprising a substantially C5 to C9 hydroconverted cut blended with a substantially C9 to C14 hydroconverted cut and a substantially C14 to C22 hydroconverted cut, which cuts have the Fischer-Tropsch process as their origin, said blend having an H:C ratio from 2.12 to 2.18.

15. A compression ignition engine, gas turbine, and fuel cell fuel which is interchangeably useable in compression ignition engines, gas turbines, and fuel cells, said fuel comprising a substantially C5 to C9 hydroconverted cut blended with a substantially C14 to C22 hydroconverted cut, which cuts have the Fischer-Tropsch process as their origin, said blend having an H:C molar ratio from 2.13 to 2.19.

16. A compression ignition engine, gas turbine, and fuel cell fuel as claimed in claim 13, having an oxidation stability of equal or less than 0.2 mg/100 ml.

17. A compression ignition engine, gas turbine, and fuel cell fuel as claimed in claim 14, having an oxidation stability of equal or less than 0.2 mg/100 ml.

18. A compression ignition engine, gas turbine, and fuel cell fuel as claimed in claim 15, having an oxidation stability of equal or less than 0.2 mg/100 ml.

19. Use of a fuel produced as claimed in claim 13, wherein when combusted the fuel yields a CO₂ emission below 3.115 gCO₂/g fuel combusted.

20. Use of a fuel produced as claimed in claim 14, wherein when combusted the fuel yields a CO₂ emission below 3.115 gCO₂/g fuel combusted

21. Use of a fuel produced as claimed in claim 15, wherein when combusted the fuel yields a CO₂ emission below 3.115 gCO₂/g fuel combusted
